(19) United States
(12) Patent Application Publication
(10) Pub. No.: US 2023/0101416 A1
(43) Pub. Date: Mar. 30, 2023

(54) FLUOROALKYL GLYCERIN DERIVATIVE AND ITS USE AS SURFACTANT

(71) Applicant: MIYOUTECH CO., LTD., Gyeonggi-do (KR)

(72) Inventors: Woong-Ku KANG, Gyeonggi-do (KR); Seok-Heon OH, Incheon (KR); Soon-Dong KWON, Gyeonggi-do (KR); Mi-Jung PARK, Gyeonggi-do (KR); Hyun-Sun AHN, Gyeonggi-do (KR); Yong-Il CHOI, Gyeongsangnam-do (KR); Ji-Hwan JEON, Gyeonggi-do (KR)

(73) Assignee: MIYOUTECH CO., LTD., Gyeonggi-do (KR)

(21) Appl. No.: 17/828,452

(22) Filed: May 31, 2022

Related U.S. Application Data
(63) Continuation of application No. PCT/KR2022/003687, filed on Mar. 16, 2022.

(30) Foreign Application Priority Data
Sep. 3, 2021 (KR) 10-2021-0117732

(51) Int. Cl. C09K 23/42; C07C 43/12

(52) U.S. Cl. CPC C09K 23/42; C07C 43/126

(57) ABSTRACT

The present disclosure discloses a fluoroalkyl glycerin derivative usable as an excellent surfactant by having functional groups and the number of carbon atoms controlled in the molecular structure, and its use as a surfactant. A fluoroalkyl glycerin derivative according to the present disclosure is used as a fluorine-based nonionic surfactant, and replaces existing fluoro compounds as well as having excellent surfactant properties compared to existing surfactants having a linear alkyl group.

### CROSS-REFERENCE TO RELATED APPLICATIONS

This application is a continuation of International Application No. PCT/KR2022/003687 filed on Mar. 16, 2022, which claims priority to Korean Patent Application No. 10-2021-0117732 filed on Sep. 3, 2021, the entire contents of which are herein incorporated by reference.

### TECHNICAL FIELD

The present disclosure relates to a fluoroalkyl glycerin derivative and its use as a surfactant.

### BACKGROUND ART

A fluorine-based surfactant is a common name for a compound in which some or all of hydrophobic groups among hydrophilic and hydrophobic groups forming a surfactant are substituted with a perfluoro group, and may be divided into cationic, anionic and nonionic according to general classification of surfactants.

A fluorine-based surfactant has excellent heat resistance and chemical stability compared to hydrocarbon-based general-purpose surfactants due to physicochemical properties of a perfluorocarbon group, and is very effective even in a strong acid concentrated alkali solution. In addition, the fluorine-based surfactant has very low interfacial tension and simultaneously exhibits hydrophobicity and oleophobicity, and therefore, is highly effective even with a very small amount.

A material including a perfluorocarbon group is known to have the lowest surface tension among currently existing materials, and it is a surfactant capable of exhibiting most superior interfacial performance among currently existing surfactants.

A fluorine-based surfactant is a surface and interface functional material, and is widely used in various fields such as semiconductors, constructions, machineries, printings and cosmetics.

Meanwhile, as a fluoro compound to provide special functions in semiconductors, fibers and coating in the field of electrical and electronics, surface treatments, surfactants, papers and various additives, PFOA (perfluorooctanoic acid) or PFOS (perfluorooctanesulfonic acid) has been used as a perfluoro compound having 8 carbon atoms. Although these compounds are very superior in their performance, the use is limited due to environmental or hazardous problems, and attempts to replace these with a fluorine-based surfactant have been made.

KR Publication No. 10-2018-0053462 discloses a hybrid-type fluorine-based nonionic surfactant having a short fluoroalkyl group and proposes that the compound is useful as a surfactant replacing a fluorine-based nonionic surfactant including a long perfluoroalkyl group such as PFOA or PFOS, however, there is a problem in that maintaining performance as a surfactant is difficult due to the substantially short fluoroalkyl group.

### RELATED ART DOCUMENT

(Patent Document 1) KR Publication No. 10-2018-0053462 (published on 2018 May 23)

### DISCLOSURE

### Technical Problem

The applicant of the present disclosure has prepared a fluoroalkyl glycerin derivative having functional groups and the number of carbon atoms controlled in the molecular structure having a new structure, and, as a result of measuring properties such as surface tension on this material, has identified that this material is usable as a nonionic surfactant.

Accordingly, the present disclosure is directed to providing a fluoroalkyl glycerin derivative and its use as a surfactant.

### Technical Solution

In view of the above, one embodiment of the present disclosure provides a fluoroalkyl glycerin derivative represented by the following Chemical Formula 1:

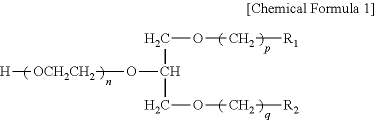

(in Chemical Formula 1, R₁, R₂, n, p and q are the same as described in the specification.)

Herein, R₁ is a C2 to C12 linear or branched alkyl group, and herein, the branched alkyl group is represented by —CH—(R₃)(R₄), and R₃ and R₄ are the same as or different from each other and are a C1 to C5 alkyl group.

In addition, the number of carbon atoms of R₃ is higher than the number of carbon atoms of R₄, and R₃ is a C3 to C5 alkyl group and R₄ is a C2 to C4 alkyl group.

In addition, R₂ is a C6 to C7 linear or branched perfluoroalkyl group.

In addition, the number of carbon atoms of R₁+R₂ is from 10 to 15.

In addition, the fluoroalkyl glycerin derivative of Chemical Formula 1 is any one of the following Chemical Formulae 2 to 9:

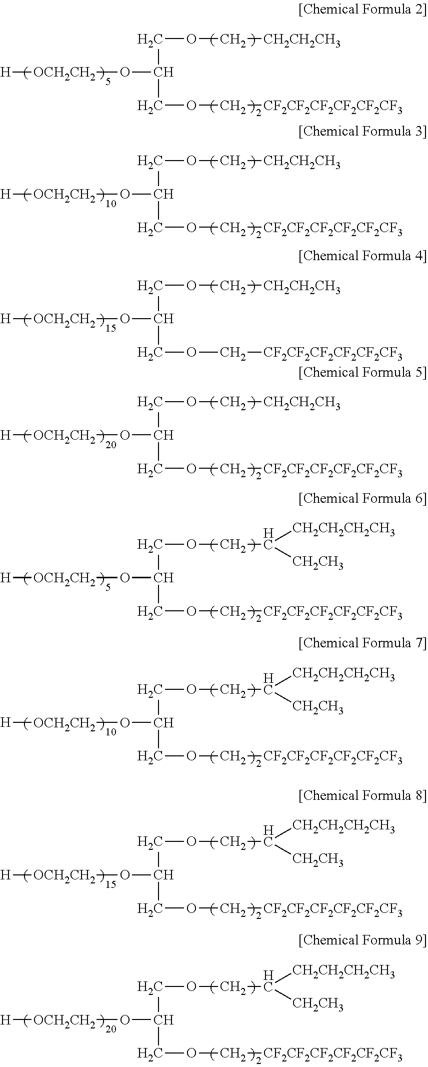

In addition, one embodiment of the present disclosure provides a fluorine-based nonionic surfactant including the fluoroalkyl glycerin derivative.

### Advantageous Effects

A fluoroalkyl glycerin derivative according to the present disclosure can be used as a fluorine-based nonionic surfactant, and can replace existing fluoro compounds as well as having excellent surfactant properties compared to existing surfactants having a linear alkyl group.

### BRIEF DESCRIPTION OF DRAWINGS

FIG. 1a is a ¹H-NMR spectrum of F6H4, FIG. 1b is a ¹⁹F-NMR spectrum thereof, FIG. 2 is a GC/MS spectrum thereof, and FIG. 3 is an FT-IR spectrum thereof.

FIG. 4 is a GC spectrum of F6H4-5EO.

FIG. 5 is a GC spectrum of F6H4-10EO.

FIG. 6 is a GC spectrum of F6H4-15EO.

FIG. 7 is a GC spectrum of F6H4-20EO.

FIG. 8a is a ¹H-NMR spectrum of F6H8, FIG. 8b is a ¹⁹F-NMR spectrum thereof, FIG. 9 is a GC/MS spectrum thereof, and FIG. 10 is an FT-IR spectrum thereof.

### BEST MODE

The present disclosure relates to a fluoroalkyl glycerin derivative represented by the following Chemical Formula 1:

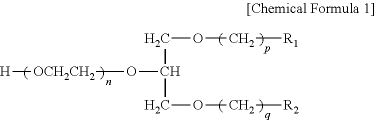

(in Chemical Formula 1, R₁, R₂, n, p and q are the same as described in the specification.)

Herein, R₁ is a C2 to C12 linear or branched alkyl group, and herein, the branched alkyl group is represented by —CH—(R₃)(R₄), and R₃ and R₄ are the same as or different from each other and are a C1 to C5 alkyl group.

In addition, the number of carbon atoms of R₃ is higher than the number of carbon atoms of R₄, and R₃ is a C3 to C5 alkyl group and R₄ is a C2 to C4 alkyl group.

In addition, R₂ is a C6 to C7 linear or branched perfluoroalkyl group.

In addition, the number of carbon atoms of R₁+R₂ is from 10 to 15.

In addition, the fluoroalkyl glycerin derivative of Chemical Formula 1 may be any one of the following Chemical Formulae 2 to 9:

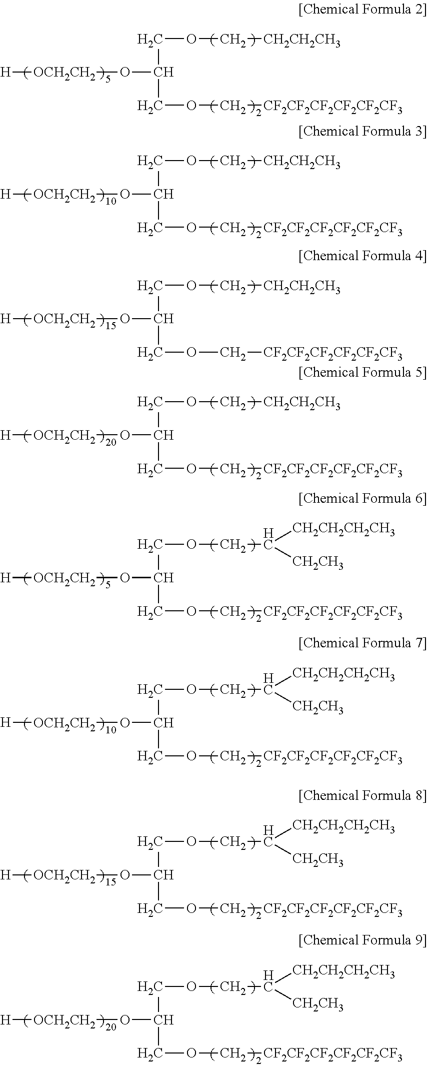

In addition, the present disclosure relates to a fluorine-based nonionic surfactant including the fluoroalkyl glycerin derivative.

### MODE FOR INVENTION

The present disclosure discloses a fluoroalkyl glycerin derivative, and its use as a fluorine-based nonionic surfactant.

The fluoroalkyl glycerin derivative having a novel structure according to the present disclosure is represented by the following Chemical Formula 1:

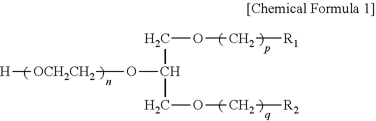

(in Chemical Formula 1,

R₁ is a C2 to C12 linear or branched alkyl group,

R₂ is a C6 to C10 linear or branched perfluoroalkyl group,

n is an integer of greater than 0 and less than or equal to 20,

p is an integer of 1 to 5, and

q is an integer of 1 to 5.)

The term ‘alkyl’ in the present disclosure means a monovalent group produced by an aliphatic saturated hydrocarbon losing one hydrogen atom. Examples of the alkyl in the present disclosure may include ethyl, n-propyl, isopropyl, n-butyl, sec-butyl, tert-butyl, n-pentyl, isopentyl, neopentyl, tert-pentyl, n-hexyl, isohexyl, octyl, decyl and the like.

The term ‘perfluoroalkyl’ in the present disclosure means that at least one (that is, one or more) hydrogen is substituted with a fluoro group, and is preferably CᵢF₂ᵢ₊₁ (herein, i is an integer of 2 to 10), particularly C₂F₅, C₃F₇, C₄F₉, C₅F₁₁, C₆F₁₃, C₇F₁₅ or C₈F₁₇ and more preferably C₆F₁₃, or partially fluorinated alkyl and particularly 1,1-difluoroalkyl, and these are all linear or branched.

According to one embodiment of the present disclosure, R₁ is a C2 to C10 linear or branched alkyl group, and herein, the branched alkyl group is represented by —CH—(R₃)(R₄), and R₃ and R₄ are the same as or different from each other and may be each independently a C1 to C5 alkyl group. More preferably, the number of carbon atoms of R₃ is higher than the number of carbon atoms of R₄, and R₃ may be a C3 to C5 alkyl group and R₄ may be a C2 to C4 alkyl group.

R₂ may be a C6 to C10, preferably a C6 to C7 linear or branched perfluoroalkyl group, and more preferably a linear perfluoroalkyl group. When the number of carbon atoms is less than the above-mentioned range, the function as a fluorine-based nonionic surfactant is not sufficient, and when the number of carbon atoms is greater than or equal to the above-mentioned range on the contrary, a problem of being harmful to the human body and the environment may occur as the number of fluorine atoms increases.

In addition, the number of carbon atoms of R₁+R₂ may preferably have a range of at least 6 or greater, more preferably 7 or greater, and most preferably 10 to 15.

Herein, n is an integer of 5 to 20 or less, p is an integer of 1 to 3, and q may be an integer of 2 to 5.

More preferably, the fluoroalkyl glycerin derivative represented by Chemical Formula 1 may be represented by the following Chemical Formulae 2 to 9, but is not limited thereto.

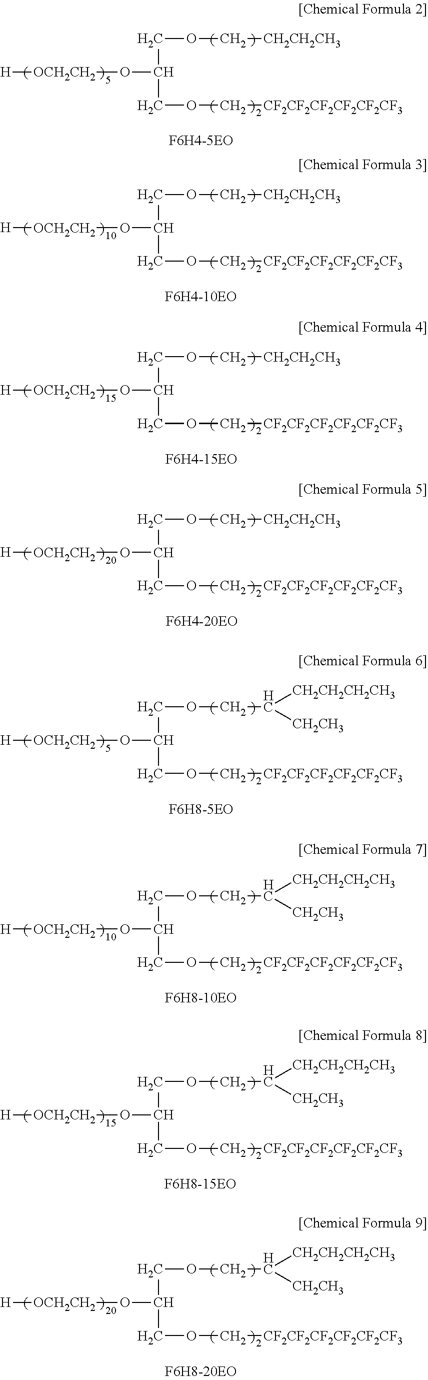

Some of the compounds of Chemical Formula 1 provided in the present disclosure contain one or more chiral centers, and accordingly, may be understood to be present in two or more stereoisomeric forms. Racemates of these isomers, individual isomers and mixtures thereof concentrated in one enantiomer, diastereomers having two chiral centers, and mixtures in which specific diastereomers are partially concentrated, and the like, are included in the scope of the present disclosure. Those skilled in the art may understand that the present disclosure includes all of individual stereoisomers (for example, enantiomers), racemic mixtures or partially decomposed mixtures of the compound of Chemical Formula 1, and properly includes individual tautomers thereof.

The fluoroalkyl glycerin derivative described above has one fluoroalkyl group and one hydrocarbon alkyl group, and, as a hybrid-type fluorine-based compound to which a polyoxyethylene group is introduced by adding ethylene oxide thereto, may be used as a fluorine-based nonionic surfactant.

By the hybrid-type fluorine-based nonionic surfactant according to the present disclosure necessarily including an unsubstituted hydrocarbon alkyl group (R₁ in Chemical Formula 1), manufacturing costs may be lowered, resulting in excellent price competitiveness.

In addition, the hybrid-type fluorine-based nonionic surfactant according to the present disclosure includes a perfluoroalkyl group (R₂ in Chemical Formula 1) in addition to the above-described unsubstituted hydrocarbon alkyl group, and, by having a limited number of perfluorinated carbon atoms, hybrid-type fluorine-based nonionic surfactant is capable of replacing an existing fluorine-based surfactant having a long perfluoroalkyl group such as PFOA (perfluorooctanoic acid) or PFOS (perfluorooctanesulfonic acid), which has been decided to be harmful to the human body and the environment.

Furthermore, the hybrid-type fluorine-based nonionic surfactant according to the present disclosure has high hydrophilicity by introducing a polyoxyethylene group thereto through an ethylene oxide addition reaction, and may be stably used as an emulsifier or a dispersant for a long period of time.

In order to evaluate performance of the hybrid-type fluorine-based nonionic surfactant including the compound represented by Chemical Formula 1 according to the present disclosure, surface tension is measured, and it is seen that the hybrid-type fluorine-based nonionic surfactant according to the present disclosure has a surface tension value enough to be used as a surfactant by exhibiting overall low surface tension, and when measuring a CMC (critical micelle concentration), the hybrid-type fluorine-based nonionic surfactant according to the present disclosure has a CMC value enough to be used as a surfactant by having an overall low CMC value. From the experimental results, it is seen that the hybrid-type fluorine-based nonionic surfactant according to the present disclosure has excellent properties as a surfactant.

In addition, when evaluating emulsification stability of the hybrid-type fluorine-based nonionic surfactant including the compound represented by Chemical Formula 1 according to the present disclosure, the hybrid-type fluorine-based nonionic surfactant according to the present disclosure has excellent emulsification stability.

As described above, the hybrid-type fluorine-based nonionic surfactant according to the present disclosure exhibits similar or more superior surfactant properties and performance compared to Comparative Example 1, a fluorine-based nonionic surfactant including a long perfluoroalkyl group used in the art, despite having a short perfluoroalkyl group, and therefore, is useful as a fluorine-based nonionic surfactant having excellent properties while being environmental-friendly and economical, and particularly, is useful as a surfactant replacing a fluorine-based nonionic surfactant including a long perfluoroalkyl group such as PFOA or PFOS known to be harmful to the environment and the human body in the art.

Accordingly, the hybrid-type fluorine-based nonionic surfactant according to the present disclosure has a short fluorine-substituted alkyl group, has low surface tension and CMC values despite including a hydrocarbon group and has very superior emulsification stability, and as a result, is useful as an environmental-friendly and economical surfactant while having excellent performance as a surfactant, and in addition thereto, is also useful as a dispersant or an emulsifier.

Meanwhile, the compound of Chemical Formula 1 according to the present disclosure is prepared by, as illustrated in the following Reaction Formula 1,

(a) preparing a compound of Chemical Formula 12 by reacting a glycidyl ether compound of Chemical Formula 10 and a perfluoroalcohol compound of Chemical Formula 11; and

(b) reacting the compound of Chemical Formula 12 and ethylene oxide of Chemical Formula 13:

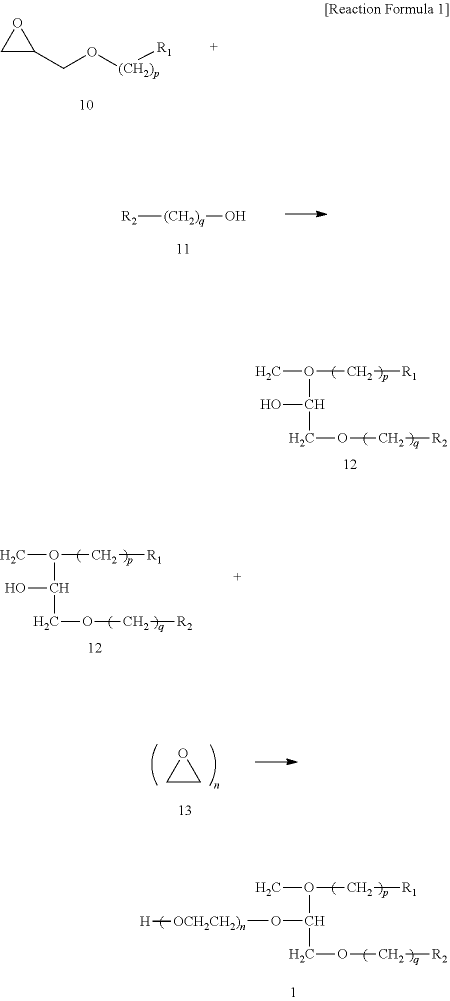

(in Reaction Formula 1, R₁, R₂, n, p and q follow the descriptions provided above.)

(Step a)

First, in the step (a), a compound of Chemical Formula 12, an intermediate, is prepared by reacting a glycidyl ether compound of Chemical Formula 10 and a perfluoroalcohol compound of Chemical Formula 11 under the presence of a base and a catalyst.

The compounds of Chemical Formula 10 and Chemical Formula 11 are reacted in a molar ratio range of 0.7 to 2:1.

According to one embodiment, the compound of Chemical Formula 10 may be, for example, glycidyl butyl ether or glycidyl 2-ethylhexyl ether.

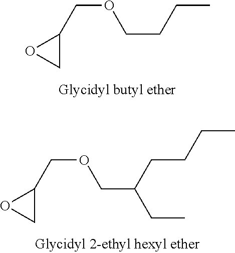

In addition, the compound of Chemical Formula 11 may be 3,3,4,4,5,5,6,6,7,7,8,8,8-tridecafluorooctan-1-ol.

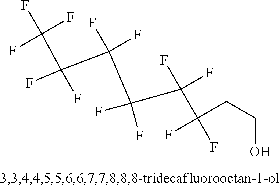 [SMILES: OCCC(F)(F)C(F)(F)C(F)(F)C(F)(F)C(F)(F)C(F)(F)F]

Herein, the reaction of the step (a) is conducted under the presence of a base and a catalyst.

As the base, an alkali metal hydroxide, an alkaline earth metal hydroxide or ammonia water may be used. Preferably, sodium hydroxide, potassium hydroxide, lithium hydroxide, calcium hydroxide, ammonia water or the like may be used, and more preferably, sodium hydroxide may be used. The base may be used in a liquid state or a solid state.

These bases are used in a molar ratio of 1.07 to 1.2 with respect to 1 mol of the compound of Chemical Formula 11. When used in less than the above-mentioned molar ratio, the reaction is insignificant and the yield is poor, and when used in greater than the above-mentioned molar ratio on the contrary, there is a problem of being not economical since an effect equal to or higher than the effect obtained when adding in an amount of the maximum molar ratio or less is not obtained.

In addition, the reaction is conducted under the presence of a polar solvent.

A solvent is a substance dissolving a solute, and is divided into a polar solvent and a non-polar solvent depending on polarity. As the non-polar solvent, hydrocarbons such as hexane and cyclohexane, or aromatic hydrocarbons such as benzene, toluene and xylene are used. Among these, aromatic hydrocarbons are used in preparing a compound such as the intermediate, and these solvents have a problem of being harmful to the human body. In addition, when using a non-polar solvent in the step (a) of the present disclosure, the reaction does not proceed well due to low solubility for the perfluoroalcohol compound of Chemical Formula 2, resulting in a low yield in preparing the compound of Chemical Formula 4. In addition, when using hexane or toluene as the solvent, there are problems in that purity is low of less than 14%, the base used may corrode equipment due to the high concentration, and the yield is impossible to check due to very low purity.

Therefore, a polar solvent is used in the present disclosure.

As the polar solvent, water; alcohols; acetates; ethers; ketones; chlorides; THF (tetrahydrofuran) and the like may be included. Among these, water, THF, and ketones such as acetone, methyl ethyl ketone (MEK) and methyl isobutyl ketone (MIBK) are used in the present disclosure. Using such a polar solvent enables preparation in a high yield such as a yield of 70% or higher.

According to one embodiment of the present disclosure, a mixed solvent of water and THF is used, and herein, water is introduced so that the base (for example, NaOH) has a concentration of 5% by weight to 40% by weight, and THF is introduced in 2 parts by weight to 4 parts by weight with respect to 1 part by weight of the perfluoroalcohol compound of Chemical Formula 11 used as a raw material.

In addition, a phase transition catalyst may be included in Reaction Formula 1, and the phase transition catalyst may be, but is not limited to, an amine-based compound or an ammonium salt-based compound, and may preferably be one or more types selected from among tetrabutylammonium bromide (TBAB), potassium hydroxide, benzyltrimethylammonium hydroxide and tetramethylammonium chloride. These catalysts are used in a molar ratio of 0.02 to 0.2 with respect to 1 mol of the perfluoroalcohol compound of Chemical Formula 11.

Herein, the reaction temperature of the step (a) is preferably from 40° C. to 100° C., and the reaction time is preferably from 6 hours to 24 hours. More preferably, the reaction temperature is from 60° C. to 65° C., and the reaction time is from 10 hours to 18 hours. When the reaction temperature is lower than 60° C., reactivity decreases and the reaction time increases thereby. When the reaction temperature is higher than 100° C., the reaction product is very likely to be discolored, side reactions increase, and the inner pressure increases due to the vapor pressure of the solvent. When the reaction time is shorter than 6 hours, produced heat is difficult to control, and when the reaction time is longer than or equal to 24 hours, side reactions increase, which reduces the yield of the product.

The compound of Chemical Formula 12 prepared as above may be separated from the reaction mixture using a proper separation means, and collected. As the separation means, common separation means such as extraction or distillation using a solvent may be used.

For example, the same amount as the water used in the reaction is used for each washing, and 5% to 10%, preferably 6% of an aqueous acetic acid solution is used once in the washing in the same amount as the water used for the washing to remove TBA, a catalyst by-product. Through this, the compound of Chemical Formula 12 may be prepared in high purity.

The step (a) may be conducted under a pressure of 0.5 atm to 50 atm, and preferably 1 atm to 15 atm.

(Step b)

In the step (b), the fluoroalkyl glycerin derivative of Chemical Formula 1 is prepared by reacting the compound of Chemical Formula 12 and a compound of Chemical Formula 13.

As for the compound of Chemical Formula 13, an ethylene oxide compound may be added in, although not particularly limited thereto, a molar number to add with respect to 1 mol of the compound represented by Chemical Formula 12, and may be preferably added in 1 mol to 20 mol and more preferably in 5 mol to 20 mol. When added in less than the above-mentioned range, there is a problem in that the prepared surfactant has poor solubility for water, and when added in greater than the above-mentioned range on the contrary, there is a problem in that there are no additional effects obtained from using an excessive amount, which is not economical.

The step (b) is also conducted under the presence of a base and a catalyst.

The base and the catalyst are used in the composition and content ranges used in the step (a).

The reaction temperature of the step (b) is preferably from 100° C. to 150° C., and the reaction time is preferably from 6 hours to 24 hours. More preferably, the reaction temperature is from 120° C. to 130° C., and the reaction time is from 10 hours to 18 hours. When the reaction temperature is lower than 100° C., reactivity decreases and the reaction time increases thereby. When the reaction temperature is higher than 150° C., the target product is very likely to be discolored, and side reactions increase. When the reaction time is shorter than 6 hours, produced heat is difficult to control, and when the reaction time is longer than or equal to 24 hours, side reactions increase, which reduces the yield of the product.

Herein, the step (b) may be conducted under a pressure of 0.5 atm to 50 atm, and preferably 1 atm to 15 atm.

The fluoroalkyl glycerin derivative of Chemical Formula 1 may be used as a fluorine-based nonionic surfactant in various fields, and may be widely used in various fields such as semiconductors, constructions, machineries, printings and cosmetics.

### EXAMPLE

Hereinafter, specific examples of the present disclosure are provided. However, the examples described below are only for specifically illustrating or describing the present disclosure, and the present disclosure is not limited thereto.

### [Example 1] Preparation of Fluoroalkyl Glycerin Derivative (F6H4-5EO)

(a) Preparation of Intermediate Alcohol (F6H4)

To a reactor equipped with a mechanical stirrer, a heater, a condenser and a thermometer, 3,3,4,4,5,5,6,6,7,7,8,8,8-tridecafluorooctan-1-ol (0.7 mol), TBAB (tetrabutylammonium bromide) (0.1 mol) and THF (500 ml) were introduced. While stirring the mixture at room temperature, an aqueous NaOH solution (500 ml, 5%) was slowly added thereto, and the result was stirred for 30 minutes.

Glycidyl butyl ether (0.5 mol) was slowly added dropwise thereto using a dropping funnel so that the inner temperature does not exceed 30° C., and the result was reacted for approximately 24 hours while stirring at 65° C. The reaction was tracked by GC (gas chromatograph), and the reaction was terminated when glycidyl butyl ether, the raw material, disappeared. After the reaction was terminated, the result was washed three times with water (100 ml), and the water layer was removed and impurities in the organic layer were removed using a 5% aqueous acetic acid solution. Then, the solvent was all removed using an evaporator, and then the result was vacuum distilled to obtain pure 1-(2,2,3,3,4,4,5,5,6,6,7,7,8,8,8-pentadecafluorooctyloxy)-3-(pentyloxy)propan-2-ol (F6H4).

Material: C₂₁H₂₆O₃F₁₇; M=494 g/mol

Yield: 70%

Purity: 72%

Appearance: yellow liquid

¹H NMR (500 MHz, CDCl₃) δ 3.94 (tt, J=6.2, 4.5 Hz, 1H, R—O—CH₂—CHOH—CH₂—O—R), 3.78 (td, J=6.7, 1.2 Hz, 2H, R—O—CH₂—R_{f}), 3.58-3.41 (m, 6H, R—CH₂—O—R), 2.42 (tt, J=18.6, 6.7 Hz, 2H, R_{f}—CF₂—CH₂—R), 1.61-1.52 (m, 2H, R—CH₂—R), 1.43-1.32 (m, 2H, R—CH₂—CH₃), 0.92 (t, J=7.4 Hz, 3H, R—CH₃).

¹⁹F NMR (471 MHz, CDCl₃) δ −81.08 (t, J=10.1 Hz, 3F, R—CF₃), −113.50 (p, J=17.1 Hz, 2F, R—CF₂CF₃), −121.92-−122.17 (m, 2F, R—CF₂—R), −123.02 (q, J=13.6, 13.1 Hz, 2F, R—CF₂—R), −123.82 (t, J=16.0 Hz, 2F, R—CF₂—R), −126.32 (td, J=15.2, 6.5 Hz, 2F, R—CH₂—CF₂—R).

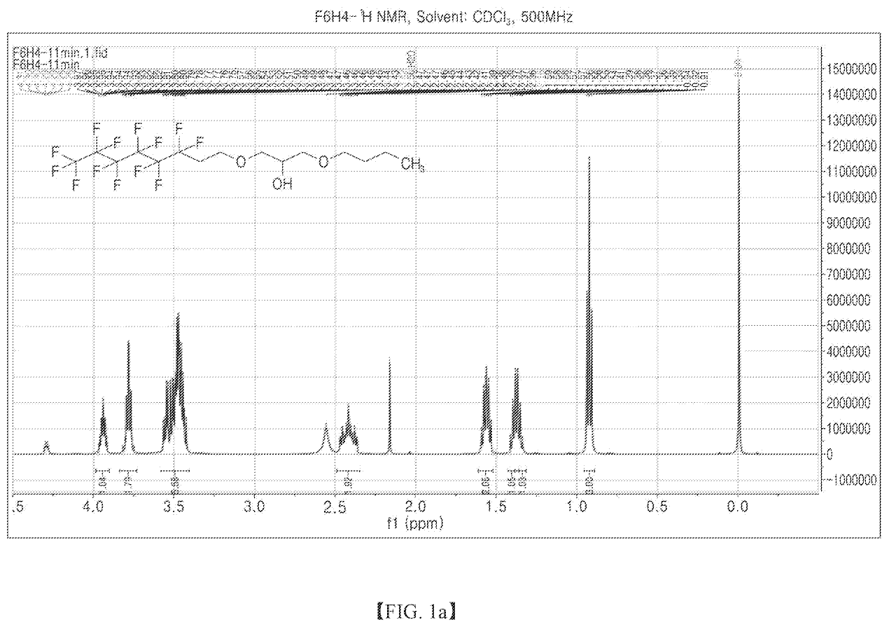

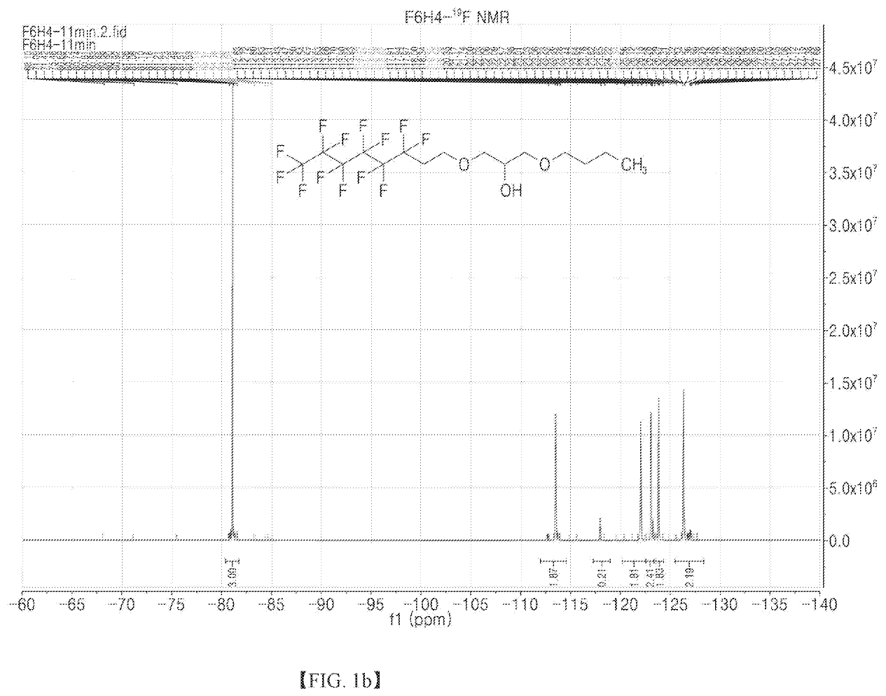

FIG. 1a is a ¹H-NMR spectrum of F6H4, FIG. 1b is a ¹⁹F-NMR spectrum thereof, FIG. 2 is a GC/MS spectrum thereof, and FIG. 3 is an FT-IR spectrum thereof. When examining FIG. 1 to FIG. 3, a compound having a molecular weight of 494 g/mol was prepared, and FT-IR peaks corresponding to C—F and OH were identified. Through identifying the OH group by the FT-IR, it is seen that conducting the ethylene oxide addition reaction in the next step (b) is possible.
NMR measuring equipment (Bruker AVANCE II+500 MHz NMR with CryoProbe Prodigy)
GC/MS measuring equipment (JEOL JMS-700)
FT-IR measuring equipment (Bruker Vertex 80v & Hyperion 2000)

(b) Preparation of Fluoroalkyl Glycerin Derivative (F6H4-5EO)

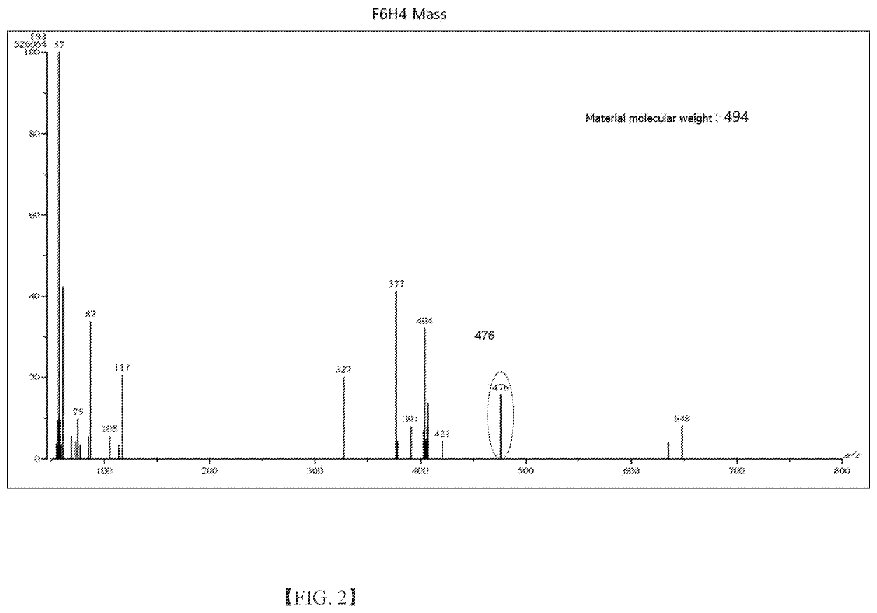

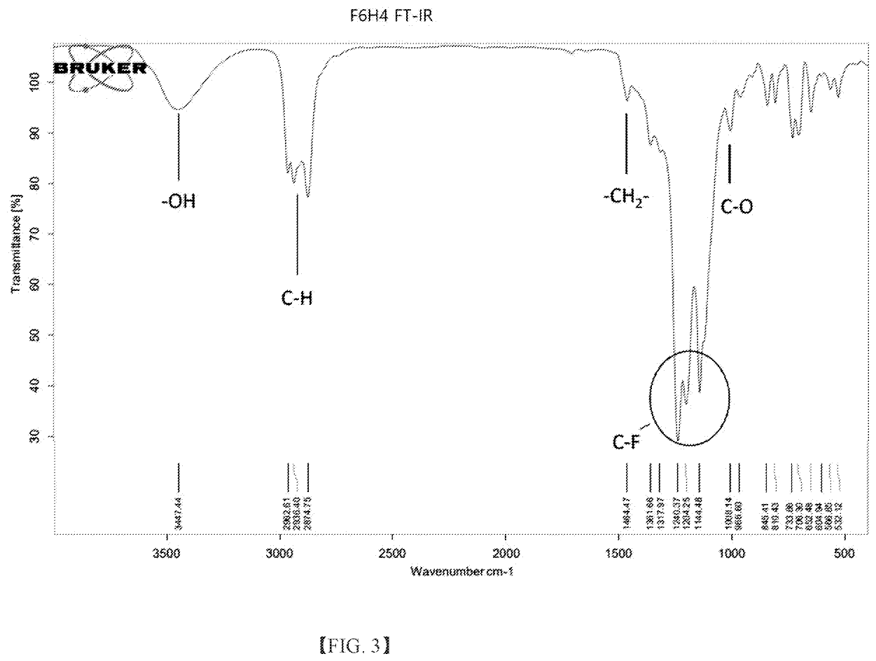

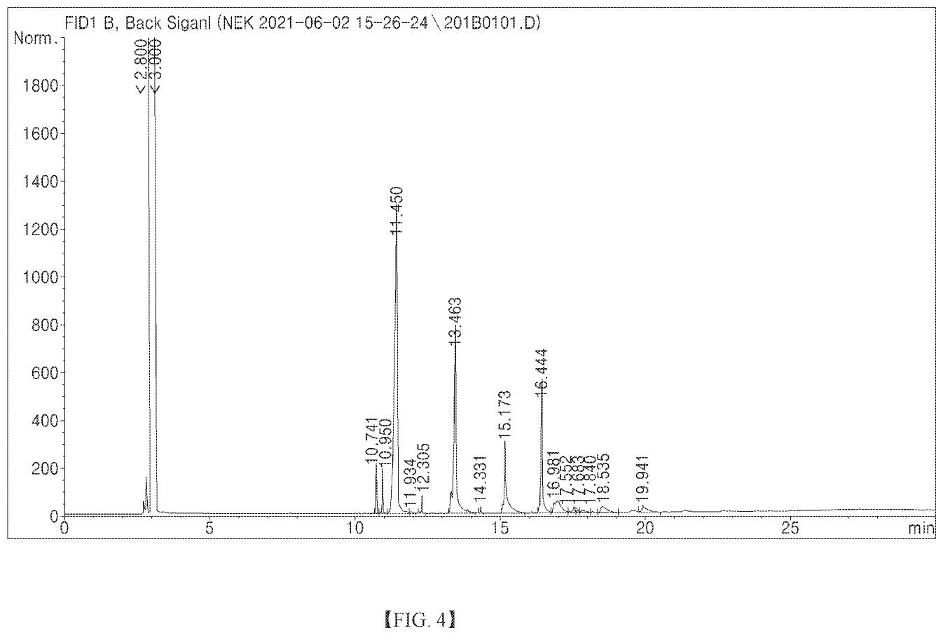

To a high-pressure reactor equipped with a stirrer, a thermometer and a cooler, 1-(2,2,3,3,4,4,5,5,6,6,7,7,8,8,8-pentadecafluorooctyloxy)-3-(pentyloxy)propan-2-ol (1 mol) obtained in the previous step and potassium hydroxide (1 mol) were added. After setting the inner temperature of the reactor to 80° C., ethylene oxide of the number of moles to add (5 mol) was introduced to the reactor, and the mixture was reacted for 4 hours at 100° C. After the reaction was terminated, the result was dissolved in chloroform (300 ml) and introduced to a separatory funnel, and then washed three times with water (150 ml). The residual organic layer was concentrated using a pressure reducing device to prepare a fluoroalkyl glycerin derivative of Chemical Formula 2. FIG. 4 is a GC spectrum of F6H4-5EO.

Weight average molecular weight: 625 g/mol

Yield: 72%

Purity: 40%

### [Example 2] Preparation of Fluoroalkyl Glycerin Derivative (F6H4-10EO)

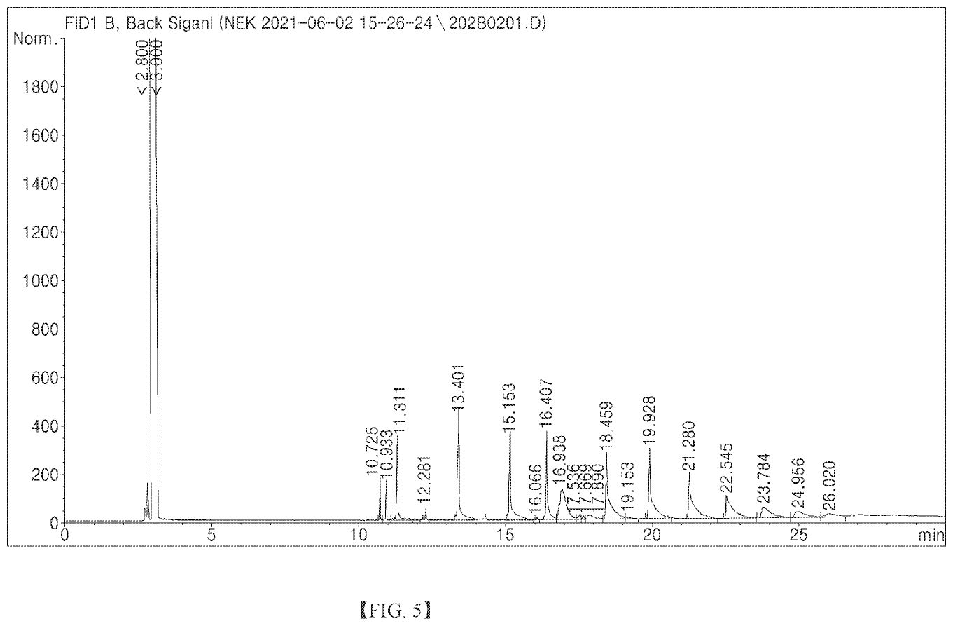

A fluoroalkyl glycerin derivative of Chemical Formula 3 was prepared in the same manner as in Example 1 except that 10 mol of ethylene oxide was added in the step (b). FIG. 5 is a GC spectrum of F6H4-10EO.

Weight average molecular weight: 787 g/mol

Yield: 75%

Purity: 75%

### [Example 3] Preparation of Fluoroalkyl Glycerin Derivative (F6H4-15EO)

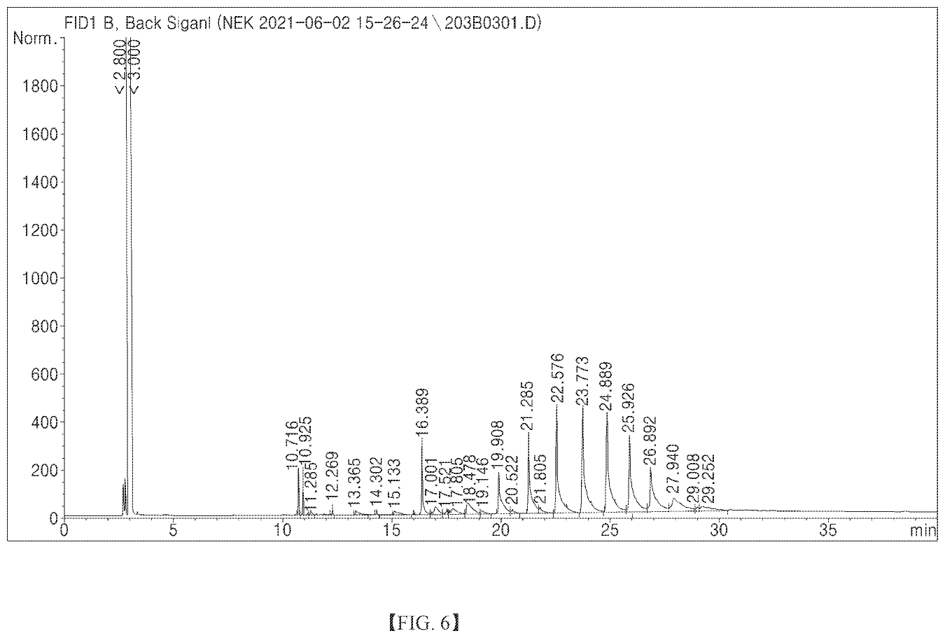

A fluoroalkyl glycerin derivative of Chemical Formula 4 was prepared in the same manner as in Example 1 except that 15 mol of ethylene oxide was added in the step (b). FIG. 6 is a GC spectrum of F6H4-15EO.

Weight average molecular weight: 852 g/mol

Yield: 86%

Purity: 94%

### [Example 4] Preparation of Fluoroalkyl Glycerin Derivative (F6H4-20EO)

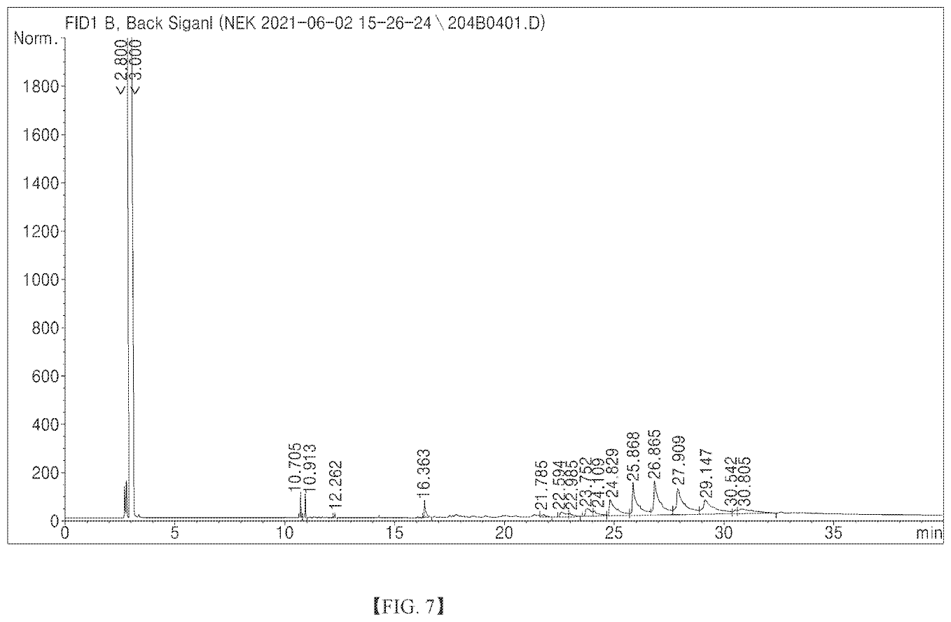

A fluoroalkyl glycerin derivative of Chemical Formula 5 was prepared in the same manner as in Example 1 except that 20 mol of ethylene oxide was added in the step (b). FIG. 7 is a GC spectrum of F6H4-20EO.

Weight average molecular weight: 15226 g/mol

Yield: 87%

Purity: 97%

### [Example 5] Preparation of Fluoroalkyl Glycerin Derivative (F6H8-5EO)

(a) Preparation of Intermediate Alcohol (F6H8)

To a reactor equipped with a mechanical stirrer, a heater, a condenser and a thermometer, 3,3,4,4,5,5,6,6,7,7,8,8,8-tridecafluorooctan-1-ol (0.7 mol), TBAB (tetrabutylammonium bromide) (0.1 mol) and THF (500 ml) were introduced. While stirring the mixture at room temperature, an aqueous NaOH solution (500 ml, 5%) was slowly added thereto, and the result was stirred for 30 minutes.

Glycidyl 2-ethylhexyl ether (0.5 mol) was slowly added dropwise thereto using a dropping funnel so that the inner temperature does not exceed 30° C., and the result was reacted for approximately 24 hours while stirring at 100° C. The reaction was tracked by GC (gas chromatograph), and the reaction was terminated when glycidyl 2-ethylhexyl ether, the raw material, disappeared. After the reaction was terminated, the result was washed three times with water (100 ml), and the water layer was removed and impurities in the organic layer were removed using a 5% aqueous acetic acid solution. Then, the solvent was all removed using an evaporator, and then the result was vacuum distilled to obtain pure 1-(2,2,3,3,4,4,5,5,6,6,7,7,8,8,8-pentadecafluorooctyloxy)-3-(2-ethylhexyloxy)propan-2-ol.

Material: C₂₁H₂₆O₃F₁₇; M=550 g/mol

Yield: 72%

Purity: 83%

Appearance: orange liquid

¹H NMR (500 MHz, CDCl₃) δ 3.94 (tt, J=6.0, 4.7 Hz, 1H, R—O—CH₂—CHOH—CH₂—O—R), 3.82-3.74 (m, 2H, R—O—CH₂—R_{f}), 3.59-3.30 (m, 6H, R—CH₂—O—R), 2.42 (tt, J=18.5, 6.8 Hz, 2H, R_{f}—CF₂—CH₂—R), 1.51 (q, J=6.1 Hz, 1H, —CH—), 1.43-1.21 (m, 8H, R—CH₂—R), 0.88 (dt, J=9.5, 7.1 Hz, 6H, R—CH₃).

¹⁹F NMR (471 MHz, CDCl₃) δ −80.93 (t, J=10.1 Hz, 3F, R—CF₃), −113.41 (p, J=17.1 Hz, 2F, R—CF₂CF₃), −121.84-−122.09 (m, 2F, R—CF₂—R), −122.93 (q, J=13.0, 12.5 Hz, 2F, R—CF₂—R), −123.73 (t, J=15.5 Hz, 2F, R—CF₂—R), −126.21 (td, J=14.8, 6.1 Hz, 2F, R—CH₂—CF₂—R).

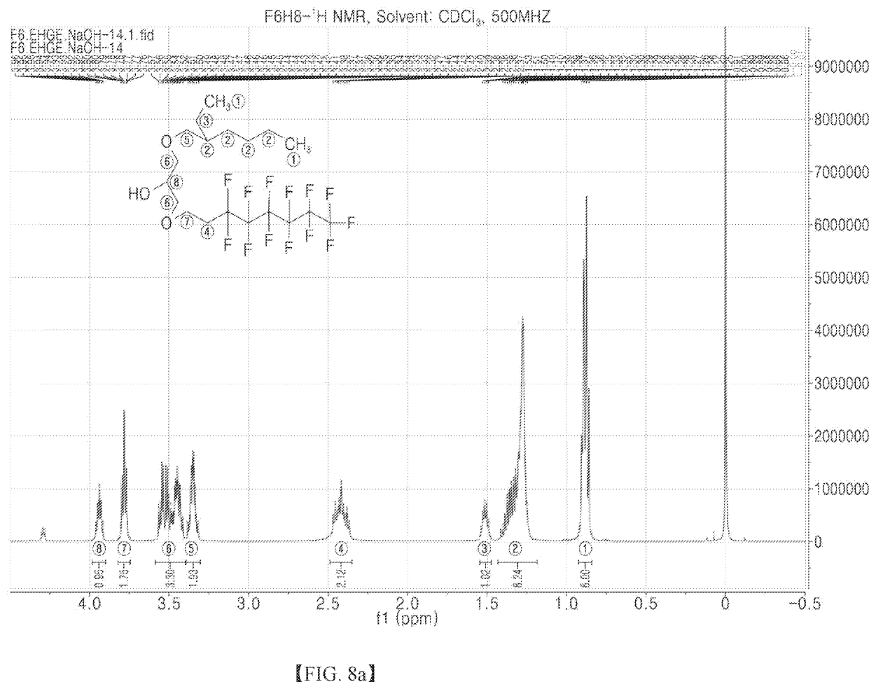

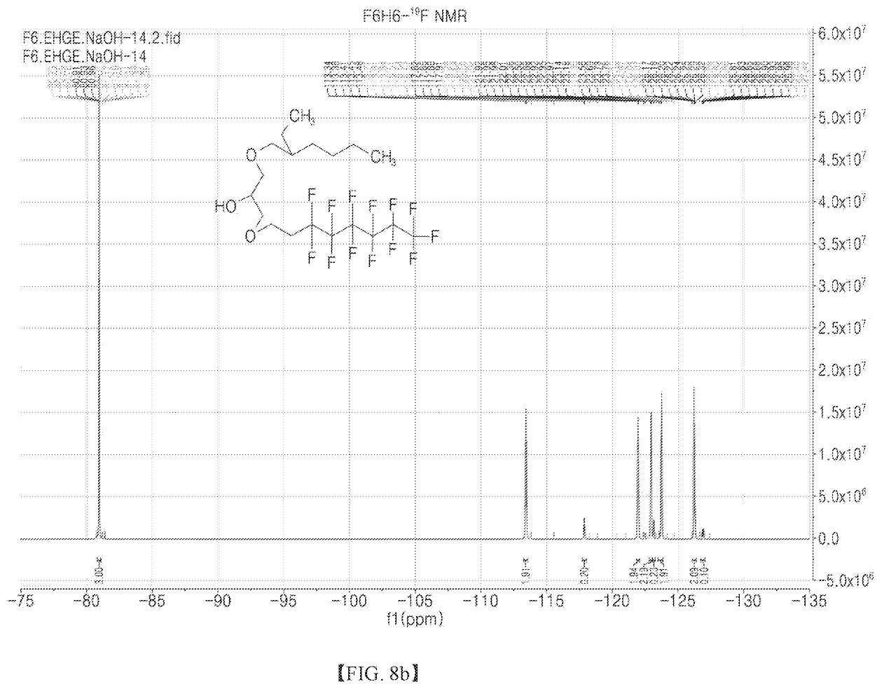

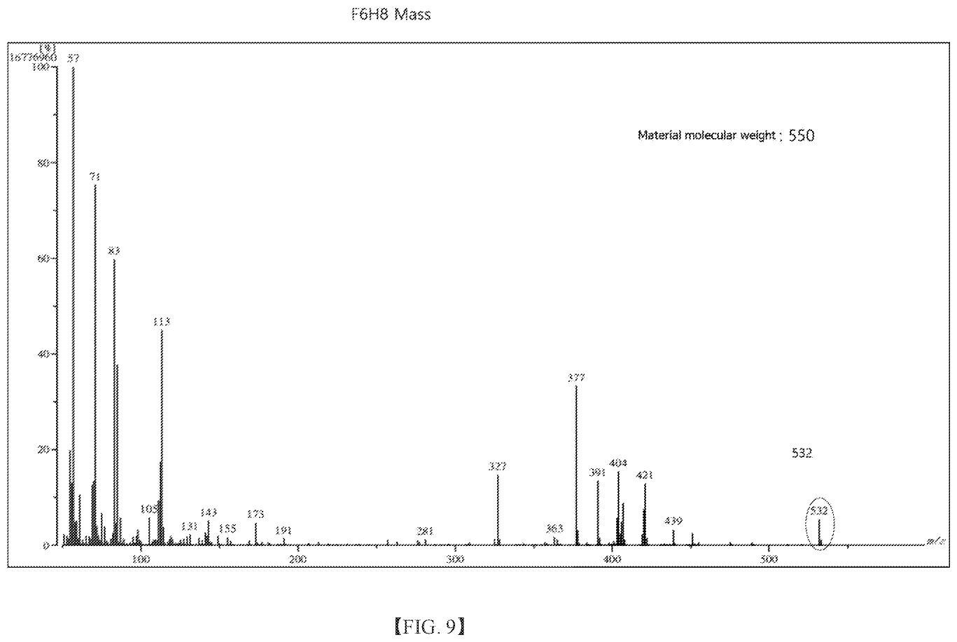

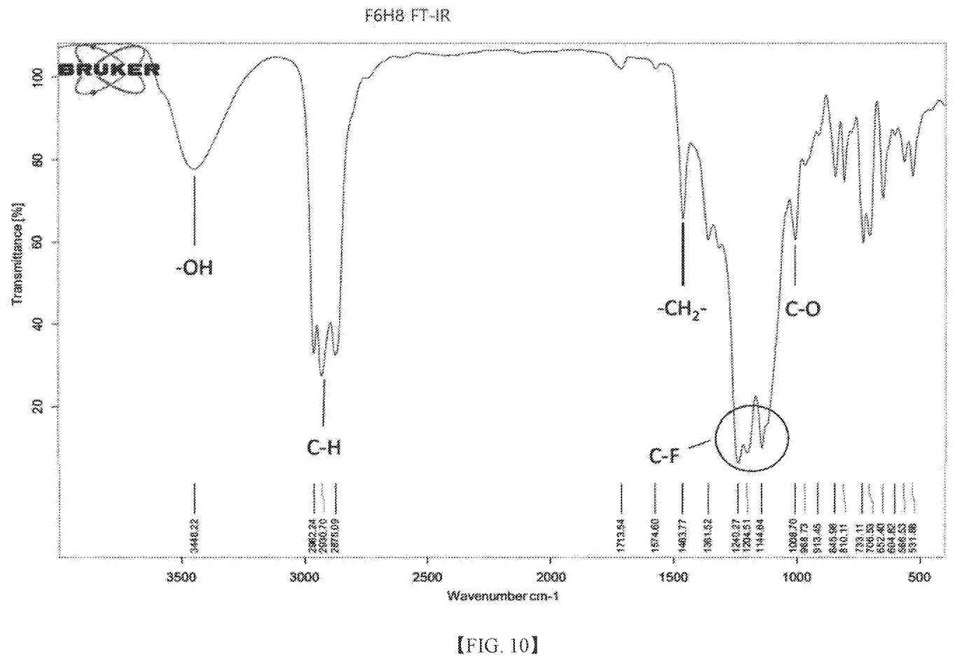

FIG. 8a is a ¹H-NMR spectrum of F6H8, FIG. 8b is a ¹⁹F-NMR spectrum thereof, FIG. 9 is a GC/MS spectrum thereof, and FIG. 10 is an FT-IR spectrum thereof. When examining FIG. 8 to FIG. 10, a compound having a molecular weight of 550 g/mol was prepared, and FT-IR peaks corresponding to C—F and OH were identified.

(b) Preparation of Fluoroalkyl Glycerin Derivative (F6H8-5EO)

To a high-pressure reactor equipped with a stirrer, a thermometer and a cooler, 1-(2,2,3,3,4,4,5,5,6,6,7,7,8,8,8-pentadecafluorooctyloxy)-3-(2-ethylhexyloxy)propan-2-ol (1 mol) obtained in the previous step and potassium hydroxide (1 mol) were added. After setting the inner temperature of the reactor to 80° C., ethylene oxide of the number of moles to add (5 mol) was introduced to the reactor, and the mixture was reacted for 4 hours at 100° C. After the reaction was terminated, the result was dissolved in chloroform (300 ml) and introduced to a separatory funnel, and then washed three times with water (150 ml). The residual organic layer was concentrated using a pressure reducing device to prepare a fluoroalkyl glycerin derivative of Chemical Formula 6.

Weight average molecular weight: 685 g/mol

Yield: 72%

Purity: 54%

### [Example 6] Preparation of Fluoroalkyl Glycerin Derivative (F6H8-10EO)

A fluoroalkyl glycerin derivative of Chemical Formula 7 was prepared in the same manner as in Example 5 except that 10 mol of ethylene oxide was added in the step (b).

Weight average molecular weight: 802 g/mol

Yield: 75%

Purity: 56%

### [Example 7] Preparation of Fluoroalkyl Glycerin Derivative (F6H8-15EO)

A fluoroalkyl glycerin derivative of Chemical Formula 8 was prepared in the same manner as in Example 5 except that 15 mol of ethylene oxide was added in the step (b).

Weight average molecular weight: 936 g/mol

Yield: 73%

Purity: 51%

### [Example 8] Preparation of Fluoroalkyl Glycerin Derivative (F6H8-20EO)

A fluoroalkyl glycerin derivative of Chemical Formula 9 was prepared in the same manner as in Example 5 except that 20 mol of ethylene oxide was added in the step (b).

Weight average molecular weight: 1857 g/mol

Yield: 78%

Purity: 74%

### Comparative Example 1

The following compound was prepared using a method corresponding to Example 12 of KR 10-2018-0053462.

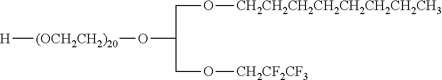

### [Experimental Example 1] Evaluation of Surface Tension

In order to evaluate properties of the fluoroalkyl glycerin derivatives obtained in Examples 1 to 8 according to the present disclosure and Comparative Example 1, surface tension was measured, and the results are shown in the following Table 1.

Surface tension was measured for an aqueous solution including each of the compounds of Examples 1 to 8 and Comparative Example 1 prepared by each concentration through PROCESSOR Tensiomneter K100 of KRUSS using a platinum ring. Herein, performance is superior as the surface tension value is lower.

TABLE 1

| | Surface Tension (mN/m) | | | | | |
|---|---|---|---|---|---|---|
| Concentration | 3% | 1% | 0.5% | 0.1% | 0.05% | 0.01% |
| Example 1 | 17.594 | 17.613 | 17.956 | 18.750 | 19.816 | 22.768 |
| Example 2 | 17.138 | 16.827 | 16.978 | 16.039 | 17.174 | 17.420 |
| Example 3 | 17.833 | 17.530 | 17.728 | 17.497 | 17.527 | 18.064 |
| Example 4 | 18.525 | 18.841 | 17.620 | 18.717 | 18.118 | 18.527 |
| Example 5 | 21.639 | 23.764 | 24.312 | 27.740 | 29.243 | 42.936 |
| Example 6 | 20.681 | 21.357 | 21.929 | 24.004 | 25.415 | 33.900 |
| Example 7 | 18.953 | 19.123 | 19.338 | 19.367 | 22.046 | 36.832 |
| Example 8 | 18.278 | 18.475 | 18.614 | 18.746 | 19.134 | 21.707 |
| Comparative | 25.126 | 26.273 | 29.254 | 30.732 | 45.125 | 52.123 |
| Example 1 | | | | | | |

As shown in Table 1, the derivatives obtained in Examples 1 to 8 according to the present disclosure had low surface tension values, and having very low surface tension compared to the fluorine-based nonionic surfactant of Comparative Example 1 was identified. In addition, it was identified that the derivative of Example 2 had a low surface tension value of 20 mN/m or less even at a low concentration of 0.01%.

Through such results, it is seen that the fluoroalkyl glycerin derivative according to the present disclosure has an excellent surface tension value even at a very low concentration, and is usable as a fluorine-based nonionic surfactant regardless of the concentration.

The present disclosure relates to a fluoroalkyl glycerin derivative usable in various fields such as semiconductors, fibers and coating in the field of electrical and electronics, surface treatments, surfactants, papers and various additives, and its use as a surfactant.

1. A fluoroalkyl glycerin derivative represented by the following Chemical Formula 1: 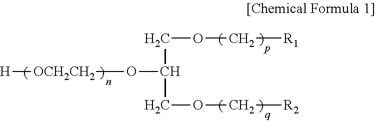
wherein, in Chemical Formula 1,
R₁ is a C2 to C12 linear or branched alkyl group;
R₂ is a C6 to C10 linear or branched perfluoroalkyl group;
n is an integer of greater than 0 and less than or equal to 20;
p is an integer of 1 to 5; and
q is an integer of 1 to 5.

2. The fluoroalkyl glycerin derivative of claim 1, wherein R₁ is a C2 to C10 linear or branched alkyl group, and herein, the branched alkyl group is represented by —CH—(R₃)(R₄), and R₃ and R₄ are the same as or different from each other and each independently a C1 to C5 alkyl group.

3. The fluoroalkyl glycerin derivative of claim 2, wherein the number of carbon atoms of R₃ is higher than the number of carbon atoms of R₄, and R₃ is a C3 to C5 alkyl group and R₄ is a C2 to C4 alkyl group.

4. The fluoroalkyl glycerin derivative of claim 1, wherein R₂ is a C6 to C7 linear or branched perfluoroalkyl group.

5. The fluoroalkyl glycerin derivative of claim 1, wherein the number of carbon atoms of R₁+R₂ is from 10 to 15.

6. The fluoroalkyl glycerin derivative of claim 1, which is any one of the following Chemical Formulae 2 to 9:

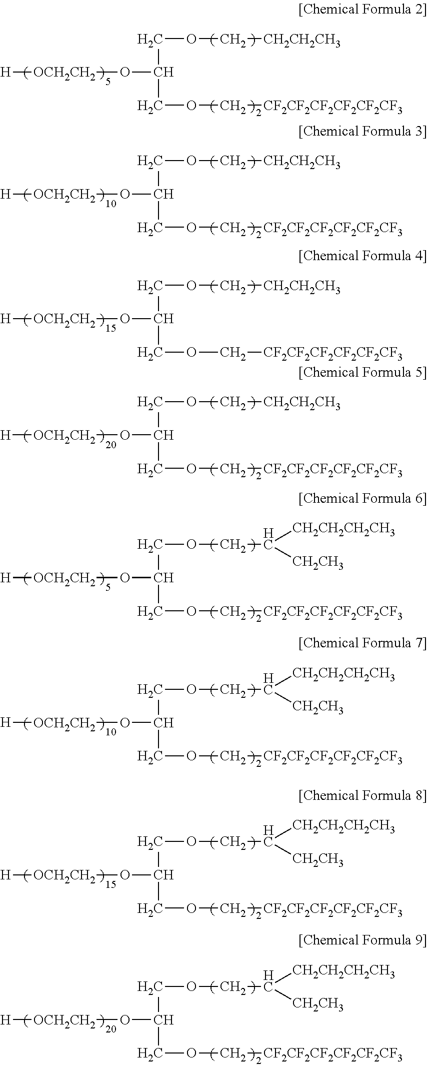

7. A fluorine-based nonionic surfactant comprising the fluoroalkyl glycerin derivative of any one of claims 1 to 6.
